(19) United States
(12) Patent Application Publication
(10) Pub. No.: US 2010/0051496 A1
(43) Pub. Date: Mar. 4, 2010

(54) Tampon Holder That Adheres To Toilet

(76) Inventor: Molly Christopher Watson, College Station, TX (US)

Correspondence Address: TOMLINSON & O'CONNELL, P.C., TWO LEADERSHIP SQUARE, 211 NORTH ROBINSON, SUITE 450, OKLAHOMA CITY, OK, 73102, US

(21) Appl. No.: 12/547,866

(22) Filed: Aug. 26, 2009

Related U.S. Application Data
(60) Provisional application No. 61/092,189, filed on Aug. 27, 2008.

(51) Int. Cl. A61B 19/00; B65D 51/04

(52) U.S. Cl. 206/440; 220/810

(57) ABSTRACT

A container for feminine hygiene products. The container is designed to hold feminine hygiene products and to be placed on the side of a toilet or other vertical surface. The container has a body compartment, a mounting assembly, a top lid, a hinge, and a latch. The body compartment has an open top and is sized to hold a variety of tampons. The lid is operatively connected to the body by the hinge and closes to cover the open top of the body. The hinge is biased to keep the lid in an open position. The latch releasably secures the top lid in the closed position. The mounting assembly, such as suction cups, allows the container to be secured to the side of a toilet. In that position, the container provides feminine hygiene products easily accessible to a female user.

### CROSS REFERENCE TO RELATED APPLICATION

This application claims the benefit of U.S. Provisional Application No. 61/092,189, filed on Aug. 27, 2008, the contents of which are incorporated herein fully by reference.

### FIELD OF THE INVENTION

The present invention relates to containers for holding feminine hygiene products.

### SUMMARY OF THE INVENTION

The present invention is directed to a container for holding feminine hygiene products. The container comprises a body compartment, a mounting assembly, a top lid, a hinge, and a latch. The body compartment defines an open top and comprises a bottom, a front wall, a back wall, and a first side wall and a second side wall. The side walls oppose each other. The mounting assembly is attached to the back wall of the body. The top lid has a first end and a second end. The hinge is operatively connected to the second side wall proximate the open top of the body and operatively connected to the second end of the lid. The top lid is movable between a closed position in which the lid covers the open top of the body and an open position in which the lid does not cover the open top of the body. The hinge is adapted to bias the top lid in the open position. The latch is adapted to releasably secure the top lid in the closed position.

### BRIEF DESCRIPTION OF THE FIGURES

FIG. 1 is a perspective view of a container built in accordance with the present invention.

FIG. 2 is a left side plan view of the container from FIG. 1.

FIG. 3 is a right side plan view of the container from FIG. 1.

FIG. 4 is a back side plan view of the container from FIG. 1.

FIG. 5 is a front side plan view of the container from FIG. 1.

FIG. 6 shows an alternative embodiment for a latch to be used with the container of FIG. 1.

FIG. 7 shows the container of FIG. 1 attached to a toilet.

### BRIEF DESCRIPTION OF THE INVENTION

Most females are aware of an approximate date when their menstrual period is coming; however, the exact start day and time is a surprise each month. This is usually discovered while on the toilet. Unless the female has shelving or storage of some kind close to the toilet, she is in a predicament as to how to obtain a tampon or sanitary napkin without getting blood on her clothes or floor. Other problems can manifest if tampons with different characteristics are not readily available.

The present invention has for its purpose to provide a container that is easily accessible and opened to expose tampons with varied characteristics—giving the user a choice of which tampon to use depending on the need of that particular female. Although the container as will be described is preferably attached to a toilet for easy access, a nearby sink, lavatory, or other surface within reach would work as well. Mounting to a toilet is superior to a wall, for instance, because: 1) many females do not have a wall within arm's length from the toilet, 2) the female user can remain seated at all times to obtain a tampon, and 3) tampons will be readily available at that unexpected time.

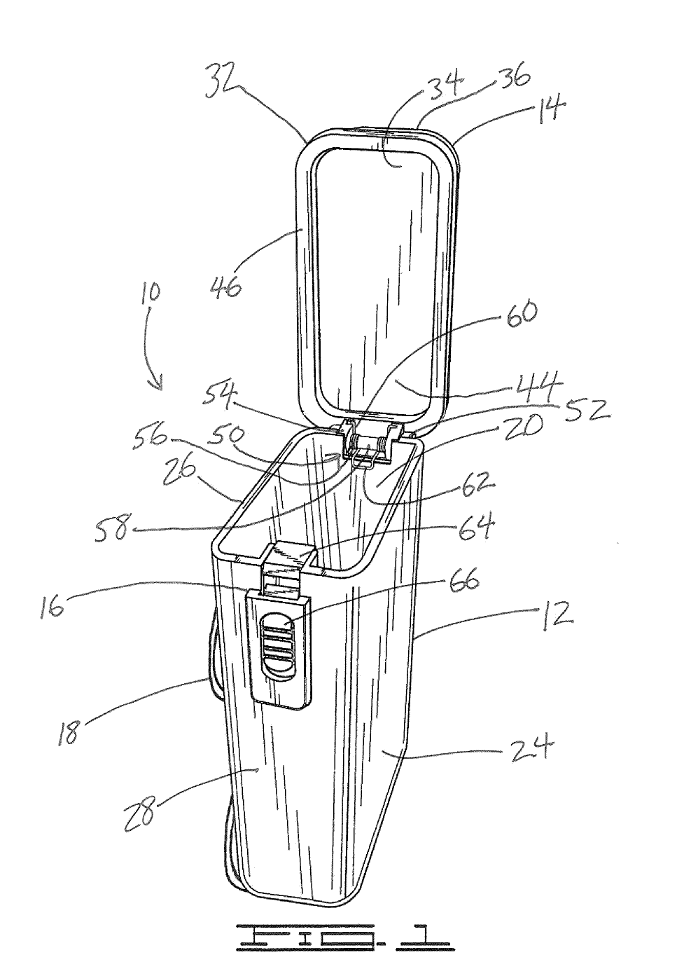

Turning now to the drawings and to FIG. 1 in particular, shown therein is a tampon box built in accordance with the present invention. The tampon box is a container 10 used to conveniently store tampons of various sizes or characteristics. The container 10 comprises a body compartment 12, a top lid 14, a latch 16, and a mounting assembly 18. Preferably the container 10 will be formed of hard molded plastic. Alternative materials are contemplated, however, such as fiberglass, paper, or recycled or other eco-friendly material.

The body compartment 12 defines an open top 20 in which a variety of tampons can be stored. The compartment 12 is comprised of a floor 22, a front wall 24, a back wall 26, and two side walls 28 and 30. The first 28 and second 30 side walls oppose each other and are each connected to the front wall 24 and the back wall 26. Securing the floor 22 to a bottom portion to each of the walls 24, 26, 28, and 30 creates the open topped compartment 12 for supporting a plurality of tampons. One skilled in the art will appreciate the use of fixed or removable dividers that may be inserted between the front wall 24 and the back wall 26 to allow for compartmentalized storage in the body 12.

Preferably, a width and length of the compartment 12 will be sized to hold at least eight (8) tampons, and preferably tampons of at least two different varieties for selection by a female user. More preferably, a height of the body compartment 12 will be less than the length of a tampon, allowing at least a portion of a tampon in the compartment to extend out of the open top 20 of the compartment. As standard tampons are approximate 4¾ inches long in their packaging, the height of the body compartment is preferably 3 inches. Allowing the body compartment 12 to have a width of 5 inches and a depth 2½ inches will allow for storage of a desired variety of tampons.

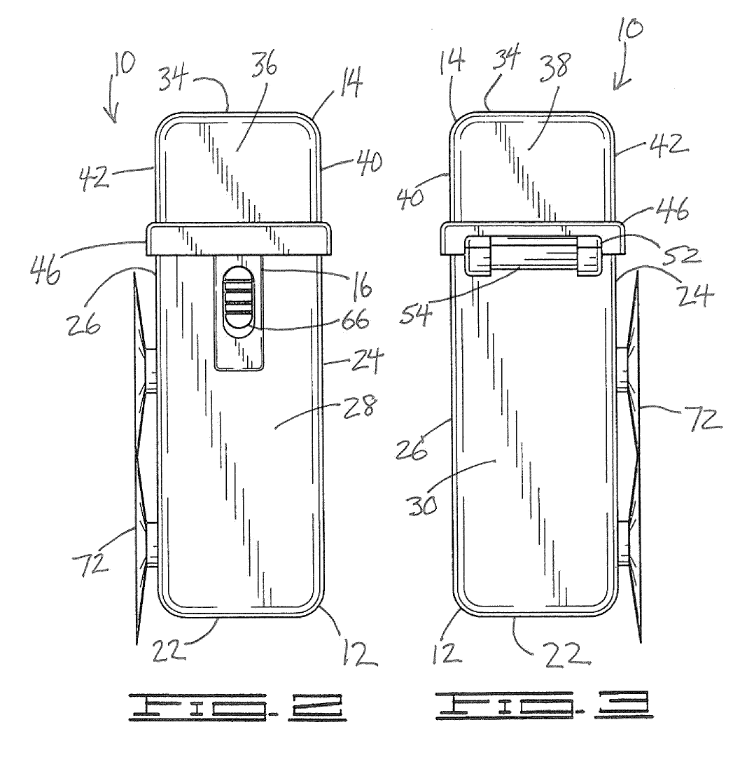

With reference now to FIGS. 1-3, the top lid 14 preferably comprises a lid compartment 32 providing a volume suitable to receive the portion of the tampon extending from the body compartment 12. The lid compartment 32 comprises a top surface 34, a front wall 36, a back wall 38, and first 40 and second 42 side walls. The side walls 40 and 42 oppose each other and are connected to the front wall 36 and the back wall 38. Securing the top surface 34 to a top portion of each of the walls 36, 38, 40, and 42 creates the lid compartment 32 that defines an open bottom 44. Preferably, the open bottom 44 of the lid compartment 32 is substantially the same size as the open top 20 of the body 12. More preferably, the lid 14 further comprises a flange 46 secured to the lid compartment 32 around a perimeter of the open bottom 44. When the lid 14 is in the closed position (shown in FIG. 2), the flange 46 rests on the perimeter of the open top 20, allowing for complete coverage of the top of the body 12. The flange 46 also provides for the body compartment 12 to be water resistant—allowing for spray cleaners to be used without getting tampons in the container 10 wet, for example.

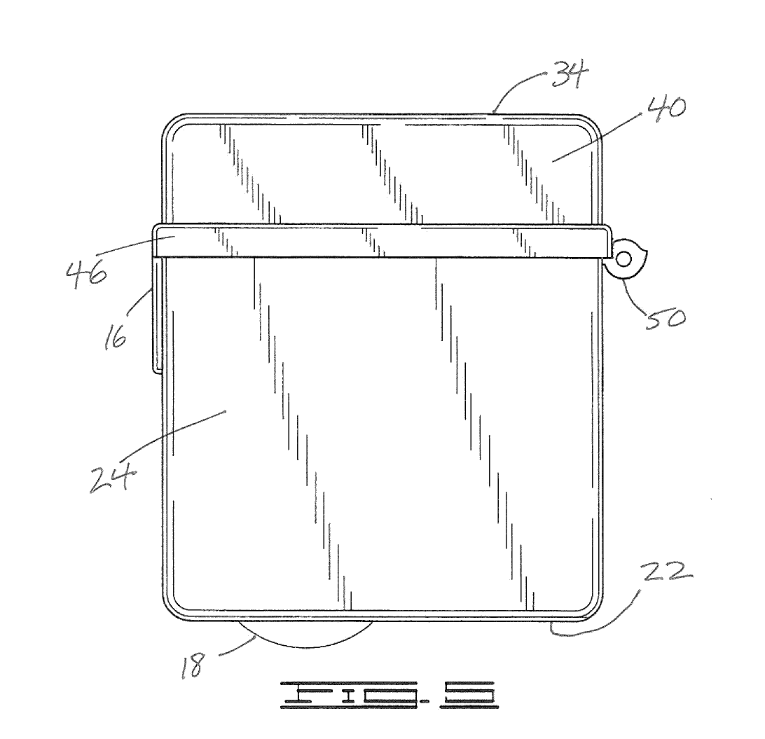

The lid 14 is movable between an open position and a closed position. In the open position, as shown in FIG. 1, the lid 14 is positioned substantially perpendicular to the body compartment 12. In the closed position, as shown in FIGS. 2-4, the lid 14 rests on the body compartment 12, covering the open top 20 of the compartment. Alternatively, the top lid 14 may be designed to slide along the perimeter of the open top 20 of the body 12 using a ridge or rail assembly along the first 28 and second 30 side walls of the body.

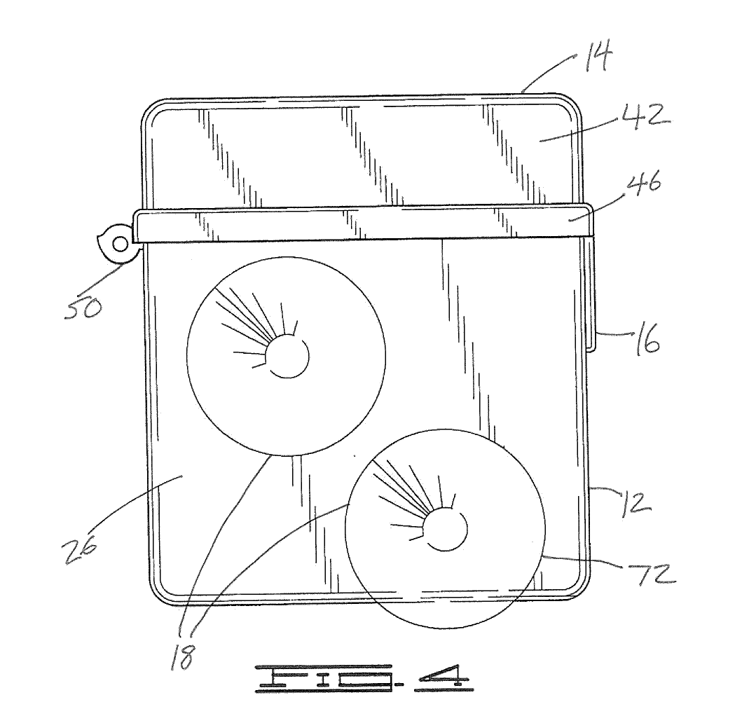

With reference now to FIGS. 1 and 3, a hinge 50 is used to pivotally connect the lid 14 to the body compartment 12. Preferably, the hinge 50 is adapted to bias the lid 14 to the open position. The hinge 50 is operatively connected to the second side wall 30 of the body 12 and the second wall 42 of the top lid 14. In this way, the top 14 is pivotally connected to the body 12 so that the top opens away from the first side wall 28. Alternative designs are contemplated so that the top 14 may pivot in a different direction. For example, the hinge 50 could be placed on the front wall 24 to allow the top 14 to pivot away from the back wall 26.

In the preferred embodiment, the second side wall 30 comprises two hinge load supports 52 secured to an exterior of the wall proximate the open top 20 of the body 12. Preferably, the load supports 52 are integrally formed with the side wall 30. The lid 14 comprises a hinge wing 54 on an exterior of the second wall 42 of the lid and positioned to fit between the load supports 52. The hinge 50 comprises a spring 56 disposed around a load pin 58. The load pin 58 is supported by load supports 52 and the hinge wing 54. The spring 52, wound around the pin 58, includes a lid contact 60 and a wall contact 62. The bias of the spring 52 operates to force the lid contact 60 and wall contact 62 to move away from each other, thus biasing the lid 14 into the open position. One skilled in the art will appreciate structure may be used to control the pivot of the top 14 to the open position. For example, rubber bands (not shown) connected between midpoints on the sides walls 28 and 30 of the body 12 and the side walls 40 and 42 of the top lid 14 may regulate the pivot motion of the top.

Referring now to FIGS. 1 and 2, the latch 16 operates to releasably secure the top lid 14 in the closed position. Preferably, the latch 16 is disposed on the first side wall 28 of the body 12 proximate the open top 20. The latch 16 comprises a lid catch 64 and a release 66. The catch 64 is disposed to engage the top lid 14 when the lid is in the closed position, maintaining the lid in the closed position. The release 66 is adapted to disengage the catch 64 from the lid 14. Preferably, the release 66 is a button or other structure that may be moved under slight pressure from the female user of the container 10. One skilled in the art will appreciate the latch 16 will allow the female user ease of use when sitting, when turning around and using two hands may be impractical. More preferably, the release 66 is biased to an up position allowing the catch 66 to engage the lid 14, and may be moved to a down position that causes the catch to disengage from the lid.

Preferably, the catch 64 and release 66 are supported within a spring-loaded housing on the first side wall 28. More preferably, the catch 64 comprises an arm having a hook at an end of the arm. The arm is biased in a latched position by a spring supported against a back side of the housing such that the hook extends above the open top 20 in a position to catch the lid 14. The release 66 button is supported against a front side of the housing and against the arm at a point above the spring. As the button is moved to a down position, the button forces the arm against the spring, causing the spring to compress and the hook to move away from and release the lid 14. When pressure on the button is released, the spring will extend against the arm and force the button back to the up position. One skilled in the art will appreciate alternative structures for the latch 16, such as electronically activated catches or rotating or pivoting catches to engage the lid.

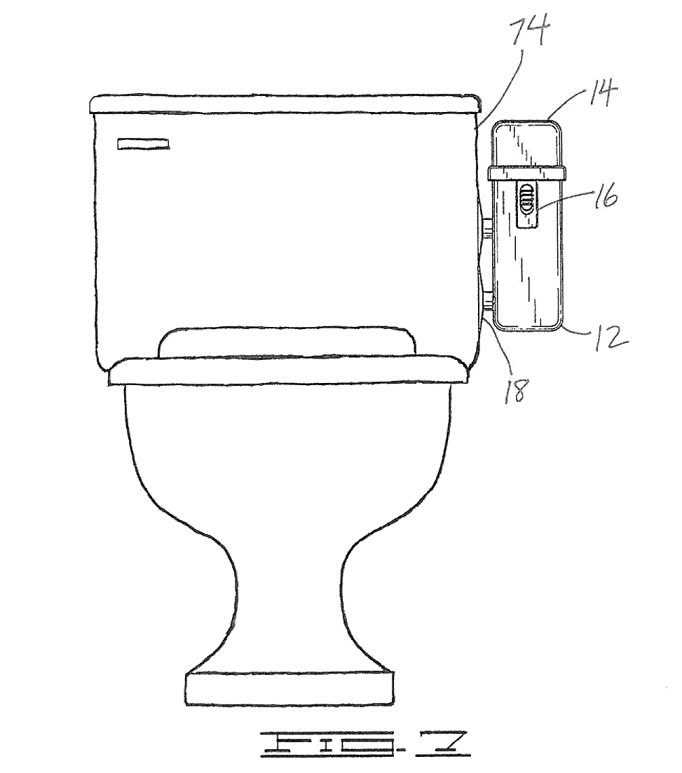

An alternative embodiment for the latch 16a is shown in FIG. 6. As shown therein, the latch 16a comprises a latch tab 68 on the first side wall 28 proximate the open top 20. A latch hook 70 is pivotally disposed on the first side wall 40 of the lid 14. The hook 70 is pivoted to latch and secure to the tab 68 when the lid is in the closed position. One skilled in the art will appreciate the latch hook 70 could be released from the tab 68 with pressure from the user's thumb, resulting in the top lid 14 moving to the open position.

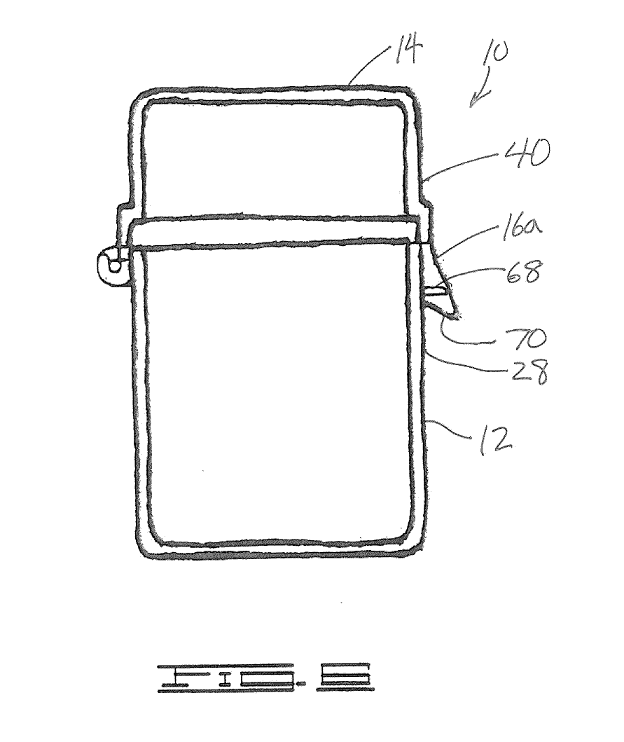

With reference now to FIGS. 2-5, the mounting assembly 18 is operatively secured to an exterior of the back wall 26 of the body compartment 12. Preferably, the mounting assembly 18 comprises at least one suction cup 72. More preferably, the assembly 18 comprises two suction cups 72 vertically separated on the back wall 26. The suction cups 72 allow for the container 10 to be easily secured to any clean dry surface, such as a lavatory, a mirror, or a wall. Preferably, the contained 10 will be secured to the side of a toilet tank. Suction cups 72 are the preferred way to mount the container 10 because they can be cleaned easily, are easily attachable, and remain secure to the composition of most toilets. One skilled in the art will appreciate other means of mounting the container 10 to a toilet, shelf, or wall, such as with brackets or Velcro.

Mounting the container 10 on the side of a toilet tank 74, as shown in FIG. 7, allows the user easy access to the tampons housed in the container. As described, the container 10 can be easily opened with one hand. The top 14 opens away from the user and parallel to the side of the tank 74 of the toilet, allowing for easy access to the tampons in the body compartment 12. The lid compartment 32 is sized so that when the top 14 is pivoted to the open position the top lid clears the top of the tampons in the body compartment 12.

Various modifications can be made in the design and operation of the present invention without departing from the spirit thereof. Thus, while the principal preferred construction of the invention has been explained in what is now considered to represent its best embodiments, which have been illustrated and described, it should be understood that the invention may be practiced otherwise than as specifically illustrated and described.

What is claimed is:

1. A container for holding feminine hygiene products, the container comprising:
a body compartment defining an open top, the body comprising:
a bottom;
a front wall;
a back wall; and
a first side wall and a second side wall, the side walls opposing each other;
a mounting assembly attached to the back wall of the body;
a top lid having a first end and a second end;
a hinge operatively connected to the second side wall proximate the open top of the body and operatively connected to the second end of the lid; and
a latch;
wherein the top lid is movable between a closed position in which the lid covers the open top of the body and an open position in which the lid does not cover the open top of the body; and
wherein the hinge is adapted to bias the top lid in the open position; and
wherein the latch is adapted to releasably secure the top lid in the closed position.

2. The container of claim 1 wherein the container is mounted to a tank of a toilet.

3. The container of claim 1 wherein the mounting assembly comprises two suction cups.

4. The container of claim 3 wherein the suction cups are disposed in a vertical relationship on the back wall.

5. The container of claim 1 wherein the second side wall comprises two hinge load supports; and
wherein the top lid comprises a hinge wing adapted to be positioned between the hinge load supports; and
wherein the hinge comprises:
a load pin; and
a spring having a lid contact and a wall contact; and
wherein the pin is secured in the load supports and passes through the hinge wing and the spring such that the lid contact of the spring is disposed to be in contact with the lid and the wall contact of the spring is disposed to be in contact with the second side wall.

6. The container of claim 1 wherein the top lid comprises a lid compartment comprising:
a top surface;
a front wall;
a back wall; and
a first side wall and a second side wall, the side walls opposing each other and connected to the front wall and the back wall;
wherein the hinge is connected to the second side wall; and
wherein the lid compartment defines an open bottom substantially the same size as the open top of the body.

7. The container of claim 6 wherein the lid further comprises a flange around a perimeter of the open bottom of the compartment, such that when the lid is in the closed position the flange rests on the perimeter of the open top of the body.

8. The container of claim 1 wherein the latch is secured to the first side wall of the body compartment.

9. The container of claim 8 wherein the latch comprises a catch and a release;
wherein the catch is adapted to engage the lid when the lid is in the closed position; and
wherein the release is adapted to disengage the catch from the lid.

10. The container of claim 1 wherein the latch is secured to the lid.

11. The container of claim 10 wherein the latch comprises a hook; and
wherein the body comprises a tab; and
wherein the hook is adapted to engage the tab when the lid is in the closed position.
